(19) United States
(12) Patent Application Publication
(10) Pub. No.: US 2023/0372272 A1
(43) Pub. Date: Nov. 23, 2023

(54) USE OF CYSTINE AND DERIVATIVES THEREOF AS ANTI-THROMBOTIC AND THROMBOLYTIC AGENTS

(71) Applicant: Georgia Tech Research Corporation, Atlanta, GA (US)

(72) Inventors: Dongjune Kim, Atlanta, GA (US); David N. Ku, Atlanta, GA (US); Susan Shea, Atlanta, GA (US)

(21) Appl. No.: 18/044,619

(22) PCT Filed: Sep. 28, 2021
(86) PCT No.: PCT/US21/52396

§ 371 Date: Mar. 9, 2023

Related U.S. Application Data
(60) Provisional application No. 63/084,305, filed on Sep. 28, 2020.

(51) Int. Cl. A61K 31/198; A61P 7/02; A61K 45/06

(52) U.S. Cl. CPC A61K 31/198; A61P 7/02; A61K 45/06

(57) ABSTRACT

The present invention provides compositions that have anti-thrombotic and thrombolytic activity. These compositions are useful, e.g., in the treatment of diseases or disorders associated with thrombus formation, such as stroke and myocardial infraction, and for other uses.

### RELATED APPLICATIONS

This application claims the benefit of priority to U.S. Provisional Application No. 63/084,305, filed Sep. 28, 2020, the contents of which are incorporated herein by reference in its entirety.

### FIELD OF THE INVENTION

Described herein compositions that have anti-thrombotic and thrombolytic activity. These compositions are useful, e.g., in the treatment of diseases or disorders associated with thrombus formation, such as stroke and/or myocardial infraction, and for other uses.

### BACKGROUND OF THE INVENTION

An acute ischemic stroke is a catastrophic event resulting from the occlusion of an artery supplying blood to the brain. Approximately 700,000 cases of ischemic stroke occur in the United States yearly, resulting in a financial burden of more than US$70 billion (Prabhakaran, S., Ruff, I. & Bernstein, R. A. Acute stroke intervention: a systematic review. JAMA 313, 1451-1462 (2015); Mozaffarian, D. et al. Heart disease and stroke statistics—2015 update: a report from the American Heart Association. Circulation 131, e29-322). Additionally, stroke survivors and their families are devastated by the disabling after effects, and many elderly patients admit to fearing survival more than death (Prabhakaran, S., Ruff, I. & Bernstein, R. A. Acute stroke intervention: a systematic review. JAMA 313, 1451-1462 (2015); Solomon, N. A., Glick, H. A., Russo, C. J., Lee, J. & Schulman, K. A. Patient preferences for stroke outcomes. Stroke 25, 1721-1725, 1994). Therefore, treatment of an acute stroke in a safe, efficacious, and expedient manner is a current major priority for the field.

The goal of ischemic stroke treatment is the expeditious clearance of the occluding thrombus to regain perfusion of the downstream vessel bed (reperfusion). The current clinical standard for stroke treatment is the use of the intravenous (IV) tissue plasminogen activator (tPA) (Prabhakaran, S., Ruff, I. & Bernstein, R. A. Acute stroke intervention: a systematic review. JAMA 313, 1451-1462, 2015; Bivard, A., Lin, L. & Parsonsb, M. W. Review of stroke thrombolytics. J Stroke 15, 90-98, 2013). tPA was first discovered in 1947 (Astrup, T. & Permin, P. M. Fibrinolysis in the Animal Organism. Nature 159, 681-682, 1947), and was tested with different types of in vitro assays (e.g. a fibrin plate (Astrup, T. & Mullertz, S. The fibrin plate method for estimating fibrinolytic activity. Archives of Biochemistry and Biophysics 40, 346-351, 1952), and a circulating plasma system (Matsuo, O., Rijken, D. C. & Collen, D. Comparison of the relative fibrinogenolytic, fibrinolytic and thrombolytic properties of tissue plasminogen activator and urokinase in vitro. Thromb Haemost 45, 225-229 (1981)). The high thrombolytic efficacy of tPA found in in vitro assays led to an in vivo study (Matsuo, O, Rijken, D. C. & Collen, D. Thrombolysis by human tissue plasminogen activator and urokinase in rabbits with experimental pulmonary embolus. Nature 291, 590-591, 1981), a small clinical study (Bergmann, S. R., Fox, K. A., Ter-Pogossian, M. M., Sobel, B. E. & Collen, D. Clot-selective coronary thrombolysis with tissue-type plasminogen activator. Science 220, 1181-1183, 1983), and large clinical trials (The GUSTO Investigators. An international randomized trial comparing four thrombolytic strategies for acute myocardial infarction. N Engl J Med 329, 673-682, 1993; The GUSTO Angiographic Investigators. The effects of tissue plasminogen activator, streptokinase, or both on coronary-artery patency, ventricular function, and survival after acute myocardial infarction. N Engl J Med 329, 1615-1622, 1993; Cohlen, D. et al. Coronary thrombolysis with recombinant human tissue-type plasminogen activator: a prospective, randomized, placebo-controlled trial. Circulation 70, 1012-1017, 1984). Still, tPA is the only Food and Drug Administration (FDA) approved thrombolytic agent in the United States (Chapman, S. N. et al. Current perspectives on the use of intravenous recombinant tissue plasminogen activator (tPA) for treatment of acute ischemic stroke. Vasc Health Risk Manag 10, 75-87, 2014). However, patients must receive treatment within 3-4.5 h of the onset of stroke symptoms, and many individuals have contraindications, such as a recent surgery or bleeding (Prabhakaran, S., Ruff, I. & Bernstein, R. A. Acute stroke intervention: a systematic review. JAMA 313, 1451-1462, 2015; Bivard, A., Lin, L. & Parsonsb, M. W. Review of stroke thrombolytics. J Stroke 15, 90-98, 2013). A clinical study found that tPA has a limited recanalization rate of less than 30% (14). Another clinical trial demonstrated a 30% relative risk reduction versus the placebo in stroke patients, but ultimately, there was no statistically significant improvement in the overall mortality with use of tPA (Chapman, S. N. et al. Current perspectives on the use of intravenous recombinant tissue plasminogen activator (tPA) for treatment of acute ischemic stroke. Vasc Health Risk Manag 10, 75-87, 2014).

tPA is a serine protease that catalyzes the conversion of plasminogen to its active form, plasmin, in the vicinity of a hemostatic plug. Plasmin then cleaves fibrin, thus breaking down or lysing the thrombus (Bivard, A., Lin, L. & Parsonsb, M. W. Review of stroke thrombolytics. J Stroke 15, 90-98, 2013; Brenner, S. The molecular evolution of genes and proteins: a tale of two serines. Nature 334, 528-530, 1988). However, thrombi causing an arterial occlusion may not be fibrin-rich, and thus tPA may not be effective in this setting. Arterial thrombi form under very high shear stress hemodynamics prior to occlusion, and are structurally very different from a fibrin gel (Ku, D. N. & Flannery, C. J. Development of a flow-through system to create occluding thrombus. Biorheology 44, 273-284, 2007). The two major contributors to occlusive stroke are ischemia from thrombotic occlusion of an atherosclerotic carotid stenosis (either in situ or thromboembolic) or the formation of embolic clots from within the heart, such as in patients with atrial fibrillation (cardioembolic) (Bivard, A., Lin, L. & Parsonsb, M. W. Review of stroke thrombolytics. J Stroke 15, 90-98, 2013). Because of the shear-dependent mechanisms of thrombus formation, these situations likely produce thrombi of very different compositions: high shear white von Willebrand Factor (VWF)-platelet thrombi in the case of a thromboembolic stroke and low shear red fibrin clots in the case of a cardioembolic stroke (Bivard, A., Lin, L. & Parsonsb, M. W. Review of stroke thrombolytics. J Stroke 15, 90-98, 2013; Friedman, M. & Van den Bovenkamp, G. J. The pathogenesis of a coronary thrombus. Am J Pathol 48, 19-44, 1966; Jorgensen, L. Experimental platelet and coagulation thrombi. A histological study of arterial and venous thrombi of varying age in untreated and heparinized rabbits. Acta Pathol Microbiol Scand 62, 189-223, 1964; Cadroy, Y., Horbett, T. A. & Hanson, S. R. Discrimination between platelet-mediated and coagulation-mediated mechanisms in a model of complex thrombus formation in vivo. J Lab Clin Med 113, 43 6-448, 1989; Para, A., Bark, D., Lin, A. & Ku, D. Rapid platelet accumulation leading to thrombotic occlusion. Ann Biomed Eng 39, 1961-1971, 2011). The composition of the clot may determine the efficacy of a thrombolytic drug. As arterial thrombi are formed under high shear conditions and are VWF-platelet rich, tPA may not be the most efficacious treatment for ischemic strokes of a thromboembolic origin. In addition, tPA has a high rate of bleeding complications due to the induction of a hyperfibrinolytic state, which deters its clinical use (Crescente, M. et al. ADAMTS13 exerts a thrombolytic effect in microcirculation. Thromb Haemost 108, 527-532, 2012; Wechsler, L. R. Intravenous Thrombolytic Therapy for Acute Ischemic Stroke. New England Journal of Medicine 364, 2138-2146, 2011; Marder, V. J. Historical perspective and future direction of thrombolysis research: the re-discovery of plasmin J Thromb Haemost 9 Suppl 1, 364-373, 2011).

Other lytic agents known in the art are likewise of questionable utility. ADAMTS-13 is a protease that cleaves VWF, the major protein responsible for capture of platelets under high shear rates, and therefore potentiation of white thrombosis (Para, A., Bark, D., Lin, A. & Ku, D. Rapid platelet accumulation leading to thrombotic occlusion. Ann Biomed Eng 39, 1961-1971, 2011; Casa, L., Gillespie, S., Meeks, S. & Ku, D. Relative contributions of von Willebrand factor and platelets in high shear thrombosis. Journal of Hematology & Thromboembolic Diseases 4, 2016; Muia, J. et al. Allosteric activation of ADAMTS13 by von Willebrand factor. Proc Natl Acad Sci USA 111, 18584-18589, 2014). Denorme et al. showed the potential use of ADAMTS-13 in acute ischemic stroke in a FeCl-induced injury mice model (Denorme, F. et al. ADAMTS13-mediated thrombolysis of t-PA-resistant occlusions in ischemic stroke in mice. Blood 127, 2337-2345, 2016). Abciximab inhibits platelet thrombus formation by blocking the glycoprotein IIb/IIIa through an antibody (Coulter Stephanie, A. et al. High Levels of Platelet Inhibition With Abciximab Despite Heightened Platelet Activation and Aggregation During Thrombolysis for Acute Myocardial Infarction. Circulation 101, 2690-2695, 2000; Kwon, O. K. et al. Intraarterially Administered Abciximab as an Adjuvant Thrombolytic Therapy: Report of Three Cases. American Journal of Neuroradiology 23, 447, 2002). N-acetylcysteine (NAC) has been shown to inhibit platelet thrombus formation via the degradation of plasma VWF multimers (Chen, J. et al. N-acetylcysteine reduces the size and activity of von Willebrand factor in human plasma and mice. J Clin Invest 121, 593-603, 2011). Furthermore, De Lizarrondo et al. demonstrated that NAC can be used in the thrombolysis of FeCl-induced thrombi (Martinez de Lizarrondo, S. et al. Potent Thrombolytic Effect of N-Acetylcysteine on Arterial Thrombi. Circulation 136, 646-660, 2017). Hastings and Ku (ISTH 2017) reported that NAC lyses platelet-rich thrombi better than do the above-mentioned thrombolytic agents (Hastings, S. M. & Ku, D. N. Dissolution of Platelet-rich Thrombus by Perfusion of N-acetyl Cysteine. 80i Research and Practice in Thrombosis and Haemostasis 1, 1-1451, 2017). However, a high variability between NAC batches has been observed.

Therefore, a great need remains for a safe and efficacious thrombolytic drug for the treatment of ischemic stroke and myocardial infarction. The compounds, compositions and methods described herein are directed towards these and other ends.

### SUMMARY OF THE INVENTION

In one aspect of the invention is provided a method of treating or preventing thrombus formation in a subject in need thereof, comprising administering to the subject cystine (e.g., L-cystine or D-cystine), or a pharmaceutically acceptable salt or derivative thereof (e.g., N,N′-diacetyl-L-cystine and/or N,N′-diacetyl-D-cystine), in an amount effective to induce thrombolysis in the subject. In certain embodiments, the thrombus is an arterial thrombus and/or a thrombus that comprises at least trace amounts of von Willebrand factor. In some embodiments, the thrombus substantially comprises von Willebrand factor and platelet cells. In certain embodiments the thrombus comprises von Willebrand factor and platelet cells, where the platelet cells have a concentration of greater than about 5%. In some embodiments, the thrombus has a paucity of red blood cells, or is substantially free of red blood cells (for example with a concentration of less than about 30% red blood cells). In other embodiments, the thrombus is substantially free of fibrin.

In some embodiments, the cystine that is administered to the subject is substantially pure and/or substantially free of N-acetylcysteine.

In some embodiments, the cystine is administered as a liquid dosage form, e.g. by intravenous injection to the subject. In some embodiments, the cystine is administered as an oral dosage form such as a tablet or liquid oral dosage form. In certain embodiments, the cystine is provided as a solution having a pH in the range of about 5 to about 8, or a pH of about 7. The cystine can be administered at any effective concentration, e.g., a concentration of about 0.5 mM to about 50 mM, or about 2 mM to about 20 mM, or about 3 mM to about 10 mM, or at a concentration of about 3 mM, or about 5 mM or about 10 mM. In some embodiments, the cystine is administered at a concentration of about 10 mM. In some embodiments, the cystine is administered in combination with a lytic agent, such as, e.g., tissue plasminogen activator (tPA), ADAMTS-13, abciximab and/or N-acetyl cysteine (NAC).

In some embodiments of the invention, the subject being administered the cystine is a human or animal subject.

In certain embodiments, the subject is suffering from a disease or disorder selected from the group consisting of: stroke, myocardial infraction, leg ischemia, a sickle-cell anemia, Disseminated Intravascular Coagulation, extracorporeal circulation, heart failure, valvular disease, aortic stenosis, and venous thrombosis.

In some embodiments, the thrombus formation occurs in a carotid artery of the subject and/or in a coronary artery of the subject and/or femoral artery of the subject and/or popliteal artery of the subject.

In certain embodiments, the treating or preventing thrombus formation results in a reduction of the diameter and/or surface area of the thrombus in an amount of at least about 50%, or about 70%, or about 95% reduction compared to a baseline value.

In another aspect is provided a method of treating or preventing a disease or disorder associated with thrombus formation in a subject in need thereof, comprising administering to the subject a therapeutically effective amount of cystine, or a pharmaceutically acceptable salt thereof.

In another aspect of the invention, is provided a method of treating or preventing thrombus formation in a cavity or device, comprising contacting the cavity or device with cystine, or a salt thereof. In certain embodiments of this aspect of the invention, the cavity or device can comprise tubing, a valve, a graft, a circuit, a stent, catheter, or a thrombectomy device. In certain embodiments, the tubing is a blood tubing. In certain other embodiments the valve is a heart valve. In certain other embodiments, the graft is a dialysis graft.

### DESCRIPTION OF THE DRAWINGS

FIG. 1 is a depiction of in vitro flow circuits used in the experiment described in Example 1.

FIG. 2 shows the in vitro perfusion system for creating platelet-rich, occlusive thrombi under arterial (high) shear rates from whole blood, followed by perfusion of lytic agents as described in Example 2. (A) Schematic of the arterial flow setup. (B) Close-up of the glass capillary tube with stenosis, which is coated with fibrillar collagen prior to perfusion. The internal dotted box denotes the region of interest. (C) Thrombus formation and subsequent perfusion with a phosphate-buffered saline (PBS) control, showing a persistent thrombus with no lysis at the end of the experiment.

FIG. 3 shows perfusions with DiNAC and NAC. Thrombus surface area was determined by pixel counting and is shown in paired images below originals, with thrombus area highlighted. (A) 2 mM DiNAC perfusion showing complete (>95% surface area reduction) lysis in 14 min (B) 20 mM DiNAC perfusion showing complete lysis in 1.5 min. (C) 2 mM NAC perfusion, with minimal lysis (<20% surface area reduction) after 60 min. (D) 20 mM NAC perfusion after 60 min, again with minimal lysis even at increased concentration. (E) Thrombus area reduction over time. Phosphate-buffered saline (PBS) is included as a negative control (black line). (F) Thrombus area after 60 min perfusion with the indicated agent (x-axis). 2 mM and 20 mM DiNAC cause significantly more lysis than the control, while neither concentration of NAC was more efficacious than PBS. DiNAC was also significantly different than NAC at each concentration. *p<0.05; **p<0.01; ***p<0.001; ****p<0.0001.

FIG. 4 shows a DiNAC dosage response with concentrations of 0.02, 0.2, 2, and 20 30 mM. The * denotes the acidic DiNAC 20 mM solution. (A) Thrombus area reduction over time. The control is shown in black. 0.02 mM DiNAC was not different from the control (square), and 2 mM DiNAC (upside down triangle) had the greatest efficacy. (B) Thrombus area after 60 min perfusion with increasing concentrations of DiNAC. Concentrations of 0.2 mM and greater were significantly different from the control. Neutralization of pH in the DiNAC solution mitigated variability and increased the surface area reduction (20 vs. 20* mM). *p<0.05; ***p<0.001; ****p<0.0001.

FIG. 5 shows thrombolysis with the other agents. Thrombus area is tPA (A), ADAMTS-13 (B), and abciximab (C) perfusion showed minimal lysis after 60 min. (D) Thrombus area reduction over time. (E) Thrombus area after 60 min perfusion with the indicated agent (x-axis). Perfusion of tPA, ADAMTS-13, and abciximab had no effect on white clot, with no differences from the control.

FIG. 6 shows lysis results on fibrin clots formed under stagnant conditions (n=3 per agent). DiNAC and NAC were tested at a concentration of 2 mM. (A) Whole blood clot lysis over 48 h. tPA showed a large decrease in clot volume. (B) PRP clot lysis over 48 h. tPA again showed a large decrease in clot volume. (C) and (D) Whole blood and PRP clot weight change over 48 h. tPA lysed clots resulting in a weight change significantly different from baseline after 6 h for whole blood and 12 h for PRP (26% and 36% reduction, respectively, **p<0.01). DiNAC, NAC, and ADAMTS-13 had no effect on red clots, with no differences from baseline nor control.

FIG. 7 shows simulation of flow and force through the stenosis using Computational Fluid Dynamics (CFD). (A) Structure of a thrombus during elongation and breakage at 0, 15, and 30 minutes of DiNAC perfusion. Black arrows denote points of tether breakage. (B) Computer model of the attached thrombus fragments at 15 min. (C) Velocity streamlines showing a jet-like flow and recirculation downstream of the elongated thrombus colored in gray. (D) Shear strain rate around the thrombus. A maximum shear of 15,000 s⁻¹ was observed in the throat of stenosis. (E) Drag force acting on the thrombi surface. The thrombus 2 fragment experiences a maximum force of >4 nN.

FIG. 8 shows the lack of thrombolytic activity of certain lytic agents tested in the experiment described in Example 3.

FIG. 9 shows the thrombolytic activity of DiNAC in the experiment described in Example 3.

FIG. 10 shows the dose response of thrombolytic activity of DiNAC in the experiment described in Example 3 at more time points.

FIG. 11 shows infusion of heparinized human whole blood through region of stenosis in 3 mm capillary tube in the experiment described in Example 4. (A) Formation of thrombus after ˜5 minutes. (B) 1 minute after infusion of 2 mM DiNAC through capillary tube. (C) 10 minutes after infusion of 2 mM DiNAC through capillary tube.

FIG. 12 is a representation of decrease in pre-stenosis pressure using DiNAC versus PBS, showing effective increase in flow through stenosis in the experiment described in Example 4.

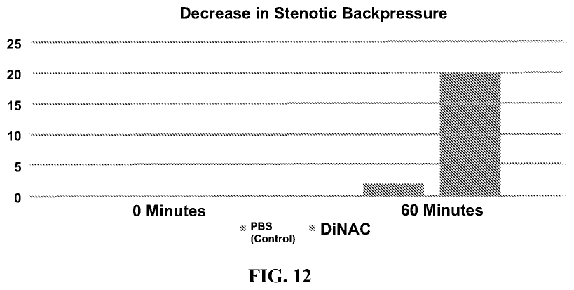

### DETAILED DESCRIPTION

The search persists for a safe and effective agent to lyse arterial thrombi in the event of an acute heart attack or strokes due to thrombotic occlusion. The culpable thrombi are composed either primarily of platelets and von Willebrand Factor (VWF), or polymerized fibrin, depending on the mechanism of formation. Current thrombolytics were designed to target red fibrin-rich clots, but are not be efficacious on white VWF-platelet-rich arterial thrombi. As described herein, Applicant has surprisingly discovered that cystine (e.g., N,N′-diacetyl-cystine; DiNAC), which is the disulfide dimer of NAC, has the potential to be a highly efficient, novel thrombolytic agent. To Applicant's knowledge, this is the first report of use of DiNAC in thrombolytic therapy and/or its use as a possible thrombolytic agent against acute arterial occlusions that could mitigate the risk of hyper fibrinolytic bleeding.

### Terms and Definitions

The term “about” refers to an amount that is near the stated amount by 10%, 5%, or 1%, including increments therein.

The term “cystine” refers to the disulfide dimer of N-acetylcysteine (NAC) and has the formula (SCH₂CH(NH₂)CO₂H)₂. Cystine is a natural amino acid, having the following structure:

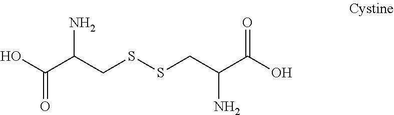 [SMILES: NC(CSSCC(N)C(=O)O)C(=O)O]

The CAS Number of cystine is 56-89-3. Also included within the term cystine is the L-enantiomer of cystine, L-cystine. The structure of L-cystine is:

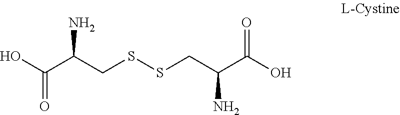 [SMILES: N[C@@H](CSSC[C@H](N)C(=O)O)C(=O)O]

Also included within the term cystine is the D-enantiomer of cystine: D-cystine. The structure of D-cystine is:

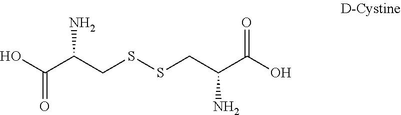 [SMILES: N[C@H](CSSC[C@@H](N)C(=O)O)C(=O)O]

The term “cystine” used herein also includes related derivatives, such as N,N′-diacetyl-cystine (DiNAC), as well as, the L- and D-enantiomers of N,N′-diacetyl-cystine, which are N,N′-diacetyl-L-cystine and N,N′-diacetyl-D-cystine, respectively. N,N′-diacetyl-cystine has the following structure:

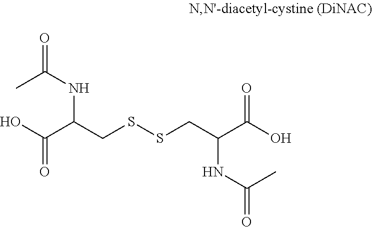 [SMILES: CC(=O)NC(CSSCC(NC(C)=O)C(=O)O)C(=O)O]

N,N′-diacetyl-L-cystine has the following structure:

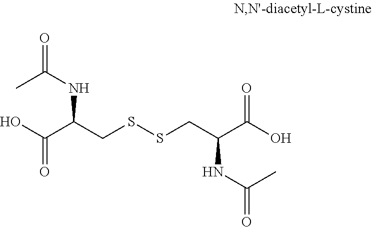 [SMILES: CC(=O)N[C@@H](CSSC[C@H](NC(C)=O)C(=O)O)C(=O)O]

N,N′-diacetyl-D-cystine has the following structure:

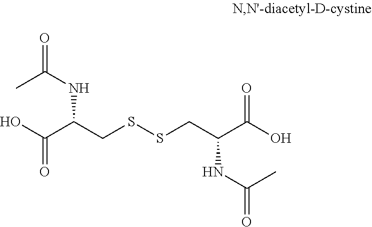 [SMILES: CC(=O)N[C@H](CSSC[C@@H](NC(C)=O)C(=O)O)C(=O)O]

As used herein, the term “cystine” also encompasses salts. As the salt form, acid addition salt, salt with base and the like can be used, and a pharmacologically acceptable salt is preferably selected. However, such salt is not particularly limited as long as it is acceptable for use in the methods of the invention. For example, salts with inorganic acid or organic acid can be used. As the inorganic acid, for example, hydrochloric acid, hydrobromic acid, nitric acid, sulfuric acid, phosphoric acid and the like can be used, and as the organic acid, formic acid, acetic acid, trifluoroacetic acid, propionic acid, lactic acid, tartaric acid, oxalic acid, fumaric acid, maleic acid, citric acid, malonic acid, methanesulfonic acid and the like are also within the scope of the invention. As the salt with a base, for example, alkali metal salts such as sodium salt, potassium salt and the like, alkaline earth metal salts such as calcium salt, magnesium salt and the like, and the like can be used.

The term “effective amount” or “therapeutically effective amount” refers to an amount effective to alleviate, delay onset of, or prevent one or more symptoms of a disease or disorder or some other condition. The “effective amount” of the formulations described herein are sufficient, when administered to a patient in need thereof, to effect treatment for disease-states, conditions, or disorders for which the compounds have utility. The amount of the formulation that constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, the drugs used in combination with or coincidentally with the compounds of the invention, and the age, body weight, general health, sex, and diet of the patient. Such a therapeutically effective amount can be determined by one of ordinary skill in the art.

The term “substantially” is to be construed as a term of approximation. The term “substantially free”, “free”, “free from”, “substantially free of”, or “free of” refers to compositions completely lacking the component or having such a small amount of the component that the component does not affect the performance of the composition. In some cases, the component may be present as an impurity or as a contaminant or as a very small amount, e.g., less than 10%, or less than 5%, or less than 0.5%. In another embodiment, the amount of the component is less than 0.1% and in yet another embodiment, the amount of component is less than 0.01%, less than 0.001%, less than 0.0001%, or less than 0.00001%.

The term “pharmaceutically acceptable” refers to compounds, carriers, excipients, compositions, and/or dosage forms that are, within the scope of sound medical judgment, suitable for use in contact with the tissues of human beings and animals without excessive toxicity, irritation, allergic response, or other problem or complication, and commensurate with a reasonable benefit/risk ratio. A pharmaceutically acceptable carrier or compound will not abrogate the biological activity or properties of the cystine and/or other components of the invention.

Percentage values described herein refer to % volume/volume percentages (v/v percent).

### Methods of Treatment and Administration

Disclosed herein are methods of treating or preventing thrombus formation in a subject in need thereof, comprising administering to the subject cystine (e.g., DiNAC), or a pharmaceutically acceptable salt thereof, in an amount affective to induce thrombolysis in the subject.

In some embodiments of the invention, the thrombus being treated, prevented or reduced is a blood clot, e.g., an aggregation of certain components, such as platelets and/or fibrin, formed, for example, in response either to an atherosclerotic lesion or to vessel or tissue injury. In some embodiments, the thrombus is an arterial thrombus (i.e., the thrombus is located in an artery). In certain embodiments, the thrombus is a white thrombus that is characterized by a predominance of platelets and/or von Willebrand Factor (VWF), and, in some cases, a paucity of red blood cells. In certain embodiments, the thrombus is substantially free of red blood cells. In some embodiments, the thrombus has a concentration of red blood cells of less than about 30%, or less than 25%, or less than 20% or less than 15% or less than 10%, or less than about 5%, or less than about 1%, or less than 0.5.

In certain embodiments, the thrombus contains at least trace amounts of von Willebrand factor. The term “von Willebrand factor” as used herein includes naturally occurring (native) VWF, but also variants thereof retaining at least some of the FVIII binding activity of naturally occurring VWF, e.g. sequence variants where one or more residues have been inserted, deleted or substituted. In some embodiments, the thrombus comprises a substantial amount of von Willebrand factor. In some embodiments, the concentration of von Willebrand factor is greater than about 0.1%, or greater than about 0.5%, or greater than about 1%, or greater than about 5%, or greater than about 10%, or greater than about 15%, or greater than about 20%, or greater than about 25%, or greater than about 30%, or greater than about 35%, or greater than about 40%, or greater than about 45%, or greater than about 50% of von Willebrand factor.

In certain embodiments, the thrombus contains at least trace amounts of platelet cells. The term “platelet” can include whole platelets, fragmented platelets, platelet derivatives, or thrombosomes. In some embodiments, the thrombus comprises a substantial amount of platelet cells. In some embodiments, the concentration of platelet cells is greater than about 0.1%, or greater than about 0.5%, or greater than about 1%, or greater than about 5%, or greater than about 10%, or greater than about 15%, or greater than about 20%, or greater than about 25%, or greater than about 30%, or greater than about 35%, or greater than about 40%, or greater than about 45%, or greater than about 50% of platelets. In certain embodiments, the thrombus has a concentration of platelets of ≤to 1% platelets.

In a particular embodiment, the platelet cells are present at a concentration of greater than about 5%.

In some embodiments, the thrombus comprises a substantial amount of von Willebrand factor and platelet cells, for example, wherein the platelet cells are present at a concentration of greater than about 5%, or greater than about 7%, or greater than about 10%, or greater than about 15%, or greater than about 20%, or greater than about 25%, or greater than about 30%, or greater than about 35%, or greater than about 40%, or greater than about 45%, or greater than about 50% of platelet cells and/or where the concentration of VWF is greater than about 0.5%, or greater than about 1%, or greater than about 5%, or greater than about 10%, or greater than about 15%, or greater than about 20%, or greater than about 25%, or greater than about 30%, or greater than about 35%, or greater than about 40%, or greater than about 45%, or greater than about 50%.

In some embodiments the thrombus is substantially free of fibrin. The term “fibrin,” as used herein refers to a fibrous protein involved in the clotting of blood. In some embodiments it is a fibrillar protein that is polymerized to form a “mesh” that forms a hemostatic plug or clot (e.g., in conjunction with platelets). Fibrin is involved in signal transduction, blood coagulation, platelet activation, and protein polymerization. In some embodiments, the fibrin has a concentration of less than about 30%, or less than 25%, or less than 20% or less than 15% or less than 10%, or less than about 5%, or less than about 1%, or less than 0.5%, or less than 0.1% or less than about 0.01%, or less than about 0.001%, or less than about 0.0001% of fibrin. However, in other embodiments, the thrombus has a substantial amount of fibrin, e.g., an amount greater than about 0.0001%, or greater than about 0.001%, or greater than about 0.01%, or greater than about 0.1%, or greater than about 1%, or greater than about 5%, or greater than about 10%, or greater than about 15% concentration of fibrin in the thrombus. In certain embodiments, the thrombus has a concentration of fibrin of </=to 1% fibrin.

Cystine is an amino acid that is found naturally in the human body, e.g., in digestive enzymes, in the cells of the immune system, in skeletal and connective tissues, skin, hair, and other areas. Cystine is a dimer of N-acetylcysteine (NAC) and has the formula (SCH₂CH(NH₂)CO₂H)₂. In some embodiments, the cystine that is administered is DL cystine. In some embodiments the cystine is L-cystine. In some embodiments the cystine is D-cystine. The scope of the disclosure also extends to derivatives of cystine that retain the desired activity and/or exhibit improved activity, and can include, e.g., cystine derivatives such as N,N′-diacetyl-cystine, including N,N′-diacetyl-L-cystine and/or N,N′-diacetyl-D-cystine, and the like. Cystine derivatives may be produced according to standard principles of medicinal chemistry, which are well known in the art. Such derivatives may also exhibit a lesser degree of activity than cystine, so long as they retain sufficient activity to be therapeutically effective. Derivatives may exhibit improvements in other properties that are desirable in pharmaceutically active agents such as, for example, improved solubility, reduced toxicity, enhanced uptake, etc.

In some embodiments, the cystine, derivatives thereof, may be present in a substantially pure or isolated form. A “substantially pure” preparation of cystine is defined as a preparation having a chromatographic purity (of the desired cystine) of greater than 50%, more preferably greater than 90%, more preferably greater than 95%, more preferably greater than 96%, more preferably greater than 97%, more preferably greater than 98%, more preferably greater than 99% and most preferably greater than 99.5% pure, as determined by area normalization of an HPLC profile. Preferably the substantially pure cystine used in the invention is substantially free of any other naturally occurring or synthetic amino acids, including amino acids that occur naturally in the human body. In this context “substantially free” can be taken to mean that no amino acids other than the target cystine are detectable by HPLC. In certain aspects of the present invention the cystine is in a synthetic form. References to cystine, particularly with regard to therapeutic use, will be understood to also encompass pharmaceutically acceptable salts of the cystine, or derivatives thereof. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

In certain embodiments of the invention, the cystine is L-cystine. In certain embodiments, the cystine is D-cystine. In certain embodiments, the cystine is N,N′-diacetyl-L-cystine and/or N,N′-diacetyl-D-cystine. In certain embodiments, the cystine is substantially pure, e.g., substantially free of other amino acids, e.g., N-acetylcysteine and/or free of impurities. In certain embodiments the cystine has a purity of at least about 50% pure, or at least about 80% pure, or about 85% pure, or about 90% pure or about 95% pure, or about 96% pure, or about 97% pure, or about 98% pure or greater than about 99% pure.

In some embodiments, the cystine can be formulated as a pharmaceutical composition further comprising one or more pharmaceutically acceptable carriers, excipients or diluents. The dosage form may contain other pharmaceutically acceptable excipients for modifying conditions such as pH, osmolarity, taste, viscosity, sterility, lipophilicity, solubility etc. The choice of diluents, carriers or excipients will depend on the desired dosage form, which may in turn be dependent on the intended route of administration to a patient. Suitable dosage forms include, but are not limited to, solid dosage forms, for example tablets, capsules, powders, dispersible granules, cachets and suppositories, including sustained release and delayed release formulations. Powders and tablets will generally comprise from about 5% to about 70% of cystine active ingredient. Solid carriers and excipients are generally known in the art and include, e.g. magnesium carbonate, magnesium stearate, talc, sugar, lactose, etc. Tablets, powders, cachets and capsules are all suitable dosage forms for oral administration. Suitable liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Liquid dosage forms also include solutions or sprays for intranasal, buccal or sublingual administration. Aerosol preparations suitable for inhalation may include solutions and solids in powder form, which may be combined with a pharmaceutically acceptable carrier, such as an inert compressed gas.

In some embodiments, the cystine is administered as a liquid dosage form, e.g., administered by intravenous administration.

In some embodiments, the cystine is administered as an oral dosage form.

In some embodiments, the cystine is administered in the form of a tablet.

In some embodiments, the cystine is administered as a solution having a pH in the range of about 3 to about 9, or about 5 to about 8, or about 7. In some embodiments, the pH of the cystine solution is neutral (i.e. around about 7).

The quantity of cystine per unit dose may be varied according to the nature of the specific form of cystine used and the intended dosage regime. Generally an effective amount shall be used, which may be within the range of from 0.01 mg to 5000 mg, preferably 0.01-4000 mg, 0.1-3000 mg, 1-2500, 5-1000, or 10-100 mg per unit dose.

In some embodiments, the cystine is administered in an amount of about 0.5 to 500 kg/day.

In some embodiments, the cystine is administered at a concentration range of about 0.01 mM to about 100 mM, or about 0.1 mM to about 90 mM, or about 1 mM to about 50 mM, or about 5 mM to about 20 mM. In some embodiments, the cystine is administered at a concentration of about 1 mM, or about 2 mM, or about 3 mM, or about 4 mM, or about 5 mM, or about 6 mM, or about 7 mM, or about 8 mM, or about 9 mM, or about 10 mM, or about 11 mM, or about 12 mM, or about 13 mM, or about 14 mM, or about 15 mM, or about 16 mM, or about 17 mM, or about 18 mM, or about 19 mM, or about 20 mM concentration.

In some embodiments, the cystine is administered at a concentration of about 0.5 mM to about 50 mM.

In some embodiments, the cystine is administered at a concentration of about 2 mM to about 20 mM.

In some embodiments, the cystine is administered at a concentration of about 3 mM to about 15 mM.

In some embodiments, the cystine is administered at a concentration of about 10 mM.

In some embodiments, the cystine is administered with other active agents, such as other pharmaceutically active ingredients. In some embodiments, the cystine is administered in combination with other lytic agents, including, but not limited to lytic agents such as tissue plasminogen activator (tPA), ADAMTS-13, abciximab and/or N-acetyl cysteine (NAC). In some embodiments, the cystine is administered in combination with more than one additional active ingredient, such as two or more lytic agents. In some embodiments the lytic agent is administered separately, sequentially or simultaneously to the cystine.

In some embodiments, the subject being administered the cystine is a human subject. In certain embodiments, the subject is an animal, such as, but not limited to, a domesticated animal (e.g., a dog or a cat or a farm animal or a mouse or a rat).

The present invention preferably aims for the treating or preventing thrombus formation in a subject, such as thrombus formation that may occur in an artery, such as a carotid artery or a coronary arty of a subject. The method of the invention further seeks to prevent and/or treat certain disorders such as stroke, myocardial infraction, leg ischemia, a sickle-cell anemia, Disseminated Intravascular Coagulation, extracorporeal circulation, heart failure, valvular disease, aortic stenosis, and venous thrombosis.

Applicant has surprisingly discovered that cystine and/or derivatives thereof (such as N,N′-diacetyl-cystine) and/or salts thereof is capable of reducing amount and/or size of thrombus to a substantial degree, and in some cases can provide a reduction of thrombus size of at least about 10%, or about 25%, or about 30% or about 35%, or about 40% or about 45% or about 50%, or about 55%, or about 60%, or about 65%, or about 70%, or about 75%, or about 80%, or about 85%, or about 86%, or about 87%, or about 88%, or about 89%, or about 90%, or about 91%, or about 92%, or about 93%, or about 94% or about 95%, or about 96% or about 97% or about 98%, or about 99% or about 100% reduction in size of thrombus compared to a baseline amount.

In some embodiments, the method of the invention provides a reduction of the size of thrombus of at least about 50%. In some embodiments, the reduction size of thrombus is at least about 70%. In some embodiments, the reduction in the size of thrombus is at least about 95%.

In some embodiments, treatment with cystine can provide a reduction of thrombus diameter of at least about 20%, or about 25%, or about 30% or about 35%, or about 40% or about 45% or about 50%, or about 55%, or about 60%, or about 65%, or about 70%, or about 75%, or about 80%, or about 85%, or about 86%, or about 87%, or about 88%, or about 89%, or about 90%, or about 91%, or about 92%, or about 93%, or about 94% or about 95%, or about 96% or about 97% or about 98%, or about 99% or about 100% reduction in diameter of thrombus compared to a baseline amount.

In some embodiments, the method of the invention provides a reduction of diameter of thrombus of at least about 50%. In some embodiments, the reduction diameter of thrombus is at least about 70%. In some embodiments, the reduction diameter of thrombus is at least about 95%.

In some embodiments, treatment with cystine and/or derivatives thereof (such as N,N′-diacetyl-cystine) and/or salts thereof is capable of reducing a surface area of thrombus by a substantial agree, e.g., by at least about 20%, or about 25%, or about 30% or about 35%, or about 40% or about 45% or about 50%, or about 55%, or about 60%, or about 65%, or about 70%, or about 75%, or about 80%, or about 85%, or about 86%, or about 87%, or about 88%, or about 89%, or about 90%, or about 91%, or about 92%, or about 93%, or about 94% or about 95%, or about 96% or about 97% or about 98%, or about 99% or about 100% reduction in surface area of thrombus compared to a baseline amount.

In some embodiments, the method of the invention provides a reduction of surface area of thrombus of at least about 50%. In some embodiments, the reduction of surface area of thrombus is at least about 70%. In some embodiments, the reduction of surface area of thrombus is at least about 95%.

In certain aspects of the invention, the method of the invention also provides a means of treating or preventing a disease or disorder associated with thrombus formation in a subject in need thereof, comprising administering to the subject a therapeutically effective amount of cystine, or a pharmaceutically acceptable salt thereof or a derivative thereof (such as N,N′-diacetyl-cystine). In certain embodiments of this aspect of the invention, the method can be used to treat any disease or disorder associated with thrombus formation, such as, for example, stroke, myocardial infraction, leg ischemia, a sickle-cell anemia, Disseminated Intravascular Coagulation, extracorporeal circulation, heart failure, valvular disease, aortic stenosis, or venous thrombosis.

In other aspects, the invention further provides a method of treating or preventing thrombus formation in a cavity or device, comprising contacting the cavity or device with cystine and/or a derivative thereof and/or a salt thereof. In certain embodiments of this aspect of the invention, the cavity or device may comprise, for example a tubing, a valve, a graft, a circuit, a stent, a catheter, or a thrombectomy device. In some embodiments, the cavity or device and/or surface comprises a tubing, that is a blood tubing, e.g., tubing that can be used to transfer blood or tubing for a blood transfusion or the like. In some embodiments, the cavity or device and/or surface comprises a valve, e.g., a heart valve such as a tricuspid valve, pulmonary valve, mitral valve, aortic valve, or an artificial valve. In some embodiments, the cavity or device and/or surface comprises a graft, such as a dialysis graft or the like.

In some embodiments, contacting the cystine and/or derivative thereof (such as N,N′-diacetyl-cystine) and/or salt thereof to a cavity or tube or body lumen or surface results in a reduction of the amount of thrombus to a substantial degree, and in some cases can provide a reduction of thrombus of at least about 10%, or about 25%, or about 30% or about 35%, or about 40% or about 45% or about 50%, or about 55%, or about 60%, or about 65%, or about 70%, or about 75%, or about 80%, or about 85%, or about 86%, or about 87%, or about 88%, or about 89%, or about 90%, or about 91%, or about 92%, or about 93%, or about 94% or about 95%, or about 96% or about 97% or about 98%, or about 99% or about 100% reduction in diameter of thrombus compared to a baseline amount.

In some embodiments, contacting the cystine and/or derivative thereof (such as N,N′-diacetyl-cystine) and/or salt thereof to a cavity or tube or body lumen or surface results in a reduction of the amount of thrombus, e.g., by at least about 10%, or about 25%, or about 30% or about 35%, or about 40% or about 45% or about 50%, or about 55%, or about 60%, or about 65%, or about 70%, or about 75%, or about 80%, or about 85%, or about 86%, or about 87%, or about 88%, or about 89%, or about 90%, or about 91%, or about 92%, or about 93%, or about 94% or about 95%, or about 96% or about 97% or about 98%, or about 99% or about 100% reduction in surface area of thrombus compared to a baseline amount.

The invention will further be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

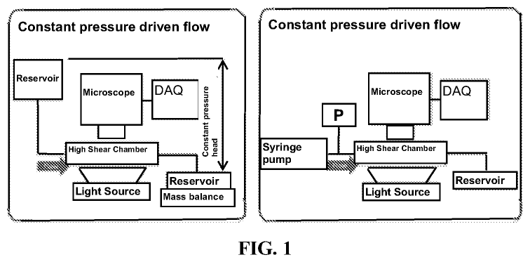

In this example, use of DiNAC and other agents as anti-thrombotic agents was tested. An illustration of the in vitro flow circuits used in this study is shown in FIG. 1. A white clot formation in the artery system was modeled by a collagen coated glass capillary tube (Ku DN, Flannery CJ. Development of a flow-through system to create occluding thrombus, Biorheology, 2007; 44(4)273-84). Porcine blood was perfused into the capillary tube using a constant pressure head or a constant flow rate (Para AN, Ku DN. A low-volume, single pass in vitro system of high shear thrombosis in a stenosis, Thrombosis Research, 2013, 131(5):418-24). After clot formation, a thrombolytic agent was perfused into the capillary tube. The detailed capillary tube experimental protocol is summarized below. Table 1 shows thrombolytic agent concentrations that were used in this experiment.

TABLE 1
Thrombolytic agent concentration.

| Agent | Concentration |
|---|---|
| tPA | 0.1 mg/mL, 0.01 mg/mL |
| ADAMTS-13 | 1 μg/mL |
| Abciximab | 3.5 μg/mL, 35 μg/mL |
| NAC | 2 mM, 20 mM |
| DINAC | 2 mM, 20 mM |
| PBS | — |

After the thrombolytic agent perfusion, the image of white clot formed in the capillary tube was observed to identify thrombolytic effect.

### Detailed Capillary Tube Experimental Protocol:

### Materials:

Glass stenotic test section (ID 1.5 mm)
Syringe pump
60 mL Luer tip syringe(s)
10 mL Luer tip syringe(s)
Small diameter tubing
Female Luer connectors
Male Luer connectors
Pressure transducer
Luer stopcock valves
Lab tape
Lab marker
Heat shrink tubing (Appendix B)
Beakers
Large petri dish
Whole blood
Phosphate-buffered saline (PBS)
Collagen
Normal saline (NaCl)
10-100 μL pipette
10-100 μL pipette tip
1.5 mL cap top centrifuge tubes
Dissecting microscope
Pco pixelfly camera
Camware or other imaging program
External storage drive
MATLAB
Laboratory stand
Clamps
Lighter
Meiji light source
Backlight platform
Tupperware container
Paper towels
Timer
Laboratory scale

### Preparation:

Select capillary tubes
Take images of the capillary tubes
Calculate % stenosis using reference ID
Label tube with a tape tag on downstream side of tube
If perfusing with syringe pump (constant Q), calculate the required flow rate given the following equation and desired initial shear rate:

$$\text{?} = \frac{4 Q}{π {r}^{3}}$$

$$\text{?} \text{indicates text missing or illegible when filed}$$

Use heat shrink tubing and lighter to affix Luer connectors to capillary tube
Cut tubing to desired lengths and affix Luer connectors

### Collagen Coat (>=24 Hours in Advance):

Prepare [(75 μL*number of test sections)=X μL] of total solution in a cap top centrifuge tubes
Prepare a 9:1 NaCl: collagen dilution
Pipette 70 μL of solution into capillary tube from the downstream side and tilt tube so that the solution rests in stenosis and downstream, but not upstream
Lay tubes with collagen solution in Tupperware container gently and ensure that solution is not disturbed and still rests in the stenosis and downstream
Soak a paper towel with hot water and wring out until damp
Put towel in with tubes and seal container
Label the container with tape: date, time of coating, and initials

### Constant Volume Flow Rate Experiment:

Assemble setup with tubing, Luer lock connectors, pressure transducer, test section, and outflow.
Fill pressure transducer with saline and close all valves to the pressure transducer
Set the flowrate on syringe pump to achieve desired shear
Turn on light source
Launch Camware:
   Use live preview and color window to position tube
   Quit live preview
   Select “Direct record to file” from drop down menu
   Select file director to save images to
   Ensure an excess of images are allocated for (e.g., 50000)
Draw 60 mL of whole blood into a 60 mL syringe from source and secure into syringe pump
Start blood flow
Begin image capture and start timer when blood contacts the stenosis
Open stopcock valve to pressure transducer and zero the transducer
Record pressure every 1 min
Stop when desired pressure head achieved

### Perfusion of Thrombus:

Prepare solution for perfusion in PBS
After the desired pressure head is obtained (to ensure perfusion capability, 25 mm Hg is safe), close the syringe port upstream of the stenosis
Attach solution syringe and set desired flow rate on syringe pump (e.g., 1 mL/min of 60 mL solution to achieve an hour of perfusion)
Begin perfusion
Open to pressure transducer and set to 25 mm Hg (viscosity decreased so pressure will be lower)
Record pressure
Stop when thrombus is gone or at desired time endpoint (e.g., 60 min)

### Constant Pressure Experiment:

Assemble setup with laboratory stand, syringe, tubing, and connectors
Ensure syringe port is dosed
Fill syringe with 60 mL blood
Set the constant height (e.g. 30 cm) based on the outlet reservoir height
Keep blood source and syringe on hand to maintain volume
Open Port
Replenish syringe to maintain volume regularly—do not exceed >5 mL decrease
Stop when the flow remarkably reduced (e.g., less than 0.1 ml/min)

### Perfusion of Thrombus:

Prepare solution for perfusion in PBS
After the desired flow rate is obtained (to ensure perfusion capability, 0.2 ml/min is safe), close the syringe port upstream of the stenosis
Attach solution syringe and set same pressure head
Begin perfusion
Record mass flow rate
Stop when thrombus is gone or at desired time endpoint (e.g., 60 min).

### Example 2

### Materials and Methods

Collagen Coating: The stenotic glass test sections (inner diameter=1.5 mm) described herein were made by the Chemistry and Biochemistry Glass Shop at Georgia Institute of Technology. The % stenosis by diameter reduction ranged from 60% to 80%. Fibrillar equine collagen (type I; Chrono-Log Corporation, Havertown, PA) was diluted 9:1 in NaCl (Sigma-Aldrich, St. Louis, MO) and incubated in the test section at the stenosis for 24 h in a warm, moist environment (Para, A., Bark, D., Lin, A. & Ku, D. Rapid platelet accumulation leading to thrombotic occlusion. Ann Biomed Eng 39, 1961-1971, 2011; Para, A. N. & Ku, D. N. A low-volume, single pass in-vitro system of high shear thrombosis in a stenosis. Thromb Res 131, 418-424, 2013).

Blood Collection: Porcine blood was collected at a local abattoir immediately following slaughter into 3.5 U/mL heparin (Thermo Fisher Scientific, Waltham, MA) or 3.2% sodium citrate (Sigma-Aldrich, St. Louis, MO). Whole blood was gently agitated using an Orbit LS shaker (Laboratory Supply Network, Atkinson, NH) until perfusion and used in experiments as soon as possible the same day as collection.

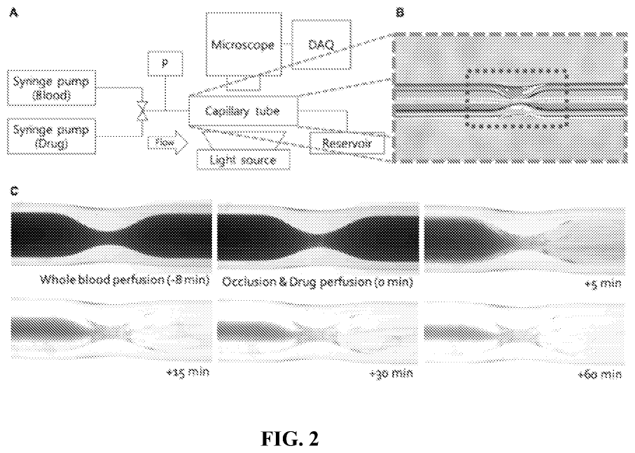

Syringe Perfusion: Lightly heparinized whole blood was perfused through the stenotic test section using a syringe pump. The flow rate was set so that the initial wall shear rate in the stenosis was 3,500 s⁻¹. A pressure transducer was connected in-line upstream of the stenosis (FIG. 2A). A dissecting microscope with a camera (PCO-Tech Incorporated, Romulus, MI) was used to capture images in real-time during perfusion. Blood perfusion continued until the upstream pressure increased by 30 mmHg (equivalent to an arterial pressure head in vivo) as a result of platelet thrombus formation in the stenotic section. Perfusion with a syringe pump (constant flow rate over constant pressure head) ensured that the thrombi were never fully occlusive, allowing subsequent perfusion for the induction of lysis.

Pharmacologic Agents: Recombinant human tPA (Sigma-Aldrich, St. Louis, MO), recombinant human ADAMTS-13 (MyBiosource, San Diego, CA), abciximab (ReoPro was kindly provided by Dr. Kevin Maher at Emory/CHOA), NAC (Thermo Fisher Scientific, Waltham, MA), and DiNAC (Cayman Chemical, Ann Arbor, Ml) or control (PBS) treatments were perfused for an hour at a flow rate of 1 ml/min. Agent solutions were made by dissolution or dilution in PBS. The in vitro flow system setup and image acquisition are shown in FIG. 2, and the concentrations of each agent are detailed in Table 2.

TABLE 2
Agent Concentration and Replicate Number.

| Agent | Concentration | Number of Replicates |
|---|---|---|
| DINAC | 0.02 mM, 0.2 mM, 2 mM, | 8, 8, 9, 8, 8 |
| | 20 mM, *20 mM | |
| NAC | 2 mM, 20 mM | 7, 5 |
| tPA | 20 g/mL | 4 |
| ADAMTS-13 | 1 g/mL | 4 |
| Acbiximab | 35 g/mL | 4 |
| PBS | — | 10 |

Real-time image capture (5 frames per second) of the stenosis continued throughout treatment perfusion.

Red clot formation and lysis: Platelet rich plasma (PRP) was made by separating citrated whole blood via gravity over a 2 h period and collecting the supernatant. Separation via gravity was employed instead of centrifugation to avoid platelet damage and activation. Citrated whole blood or PRP was then recalcified with CaCl₂ to a final [Ca²⁺] of 10 mM (Griffin, M. T., Kim, D. & Ku, D. N. Shear-induced platelet aggregation: 3D-grayscale microfluidics for repeatable and localized occlusive thrombosis. Biomicrofluidics 13, 2019), and 200 μl was transferred into 500 μal centrifuge tubes and allowed to clot and retract for 30 min. The clot was then incubated with 100 μl of either agent or control solution. The treatment solution was exchanged by removing the top 100 μl and replacement with fresh solution at 3, 6, 12, 24, and 48 h of incubation. The weight of the tube was measured immediately post-clot formation and at each timepoint after removal of old solution and prior to the replacement with fresh solution.

Computational Fluid Dynamics (CFD) Analysis: Computational fluid dynamics (CFD) was used to calculate the drag force acting on the thrombi formed in the stenosis under flow. Simulations were performed using Ansys 19.1 (Ansys Inc., PA, USA). Whole blood was assumed to be Newtonian fluid of 3.5 cP and flow was presumed as laminar, incompressible, steady, continuous, and isothermal due to the low Reynolds number (Re=16). The capillary tube was modeled with no-slip walls and 1 ml/min flow rate was applied at the inlet with zero-pressure at the outlet, reflecting experimental conditions. Mesh convergence was achieved at 3.8 million tetrahedral cells yielding a residual error of 10-9.

Data Analysis: The thrombi surface area (which was colored green) was calculated using manual pixel counting in the open-source GNU Image Manipulation Program (GIMP, Version 2.10.8, 1995-2018). Surface area reduction was calculated by % pixel reduction versus the occlusion image. Analysis of variance (ANOVA) was used to test for statistical differences between groups, with the significance set at p<0.05. Data are displayed as mean with error bars denoting standard error of the mean (SEM).

### Results

### DiNAC is a More Efficacious Thrombolytic Agent Than NAC Against Arterial White Clots:

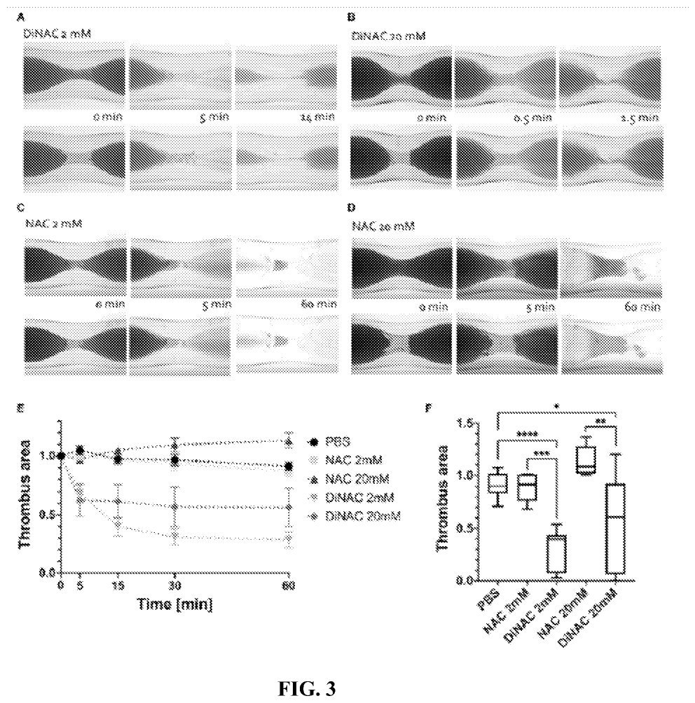

In this example, a stenotic capillary tube model, which potentiates VWF- and platelet-rich white thrombi in an arterial setting on a collagen-coated stenosis was used (Para, A., Bark, D., Lin, A. & Ku, D. Rapid platelet accumulation leading to thrombotic occlusion. Ann Biomed Eng 39, 1961-1971, 2011; Ku, D. N. & Flannery, C. J. Development of a flow-through system to create occluding thrombus. Biorheology 44, 273-284, 2007; Para, A. N. & Ku, D. N. A low-volume, single pass in-vitro system of high shear thrombosis in a stenosis. Thromb Res 131, 418-424, 2013) to test lysis of 90% occlusive white thrombi (FIG. 2) and perfused low (2 mM) and high (20 mM) concentrations of NAC and DiNAC solutions. As illustrated in FIG. 2. DiNAC, but not NAC, achieved thrombolysis over 60 minutes of perfusion (FIG. 3A-3D). 2 mM and 20 mM DiNAC lysed most of the thrombi (>95%) within 14 min and 1.5 min, respectively (FIG. 3A-3B). In contrast, NAC perfusion resulted in minimal lysis (FIG. 3C-3D). DiNAC showed a significantly higher thrombus surface area reduction than NAC after 60 min of perfusion (FIG. 3E-3F) for both concentrations (2 mM p<0.001, 20 mM, p<0.01).

### DiNAC Dose Response:

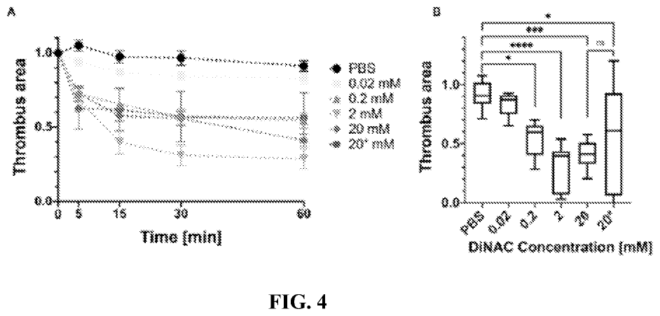

To confirm the efficacy of DiNAC, the dose response was tested at increasing concentrations of 0.02, 0.2, 2, and 20 mM. The thrombus area reduction was dependent on the concentration of DiNAC (FIG. 4). The reduction was the highest at 2 mM (7 1±20%) and slightly decreased at the highest concentration of 20 mM (59±12%), though this was not significantly different (p=0.86). Perfusion with the lowest concentration of 0.02 mM DiNAC was not significantly different from the control, but 0.2 mM showed a significantly decreased thrombus area (46±15%, p=0.02 <0.05). It was also found that the 20 mM DiNAC solution was acidic (pH-2), while 2 mM DiNAC was neutral (pH=7). Considering the practical use of the DiNAC as a thrombolytic in a clinical setting, the 20 mM DiNAC with sodium bicarbonate to a final pH=7. Neutralization of 20 mM DiNAC reduced the variance from 47% to 12% in surface area reduction (44±47% vs 59±12%, p=0.77, FIG. 4).

### The Effect of Other Thrombolytic Agents on Platelet-Rich White Clot:

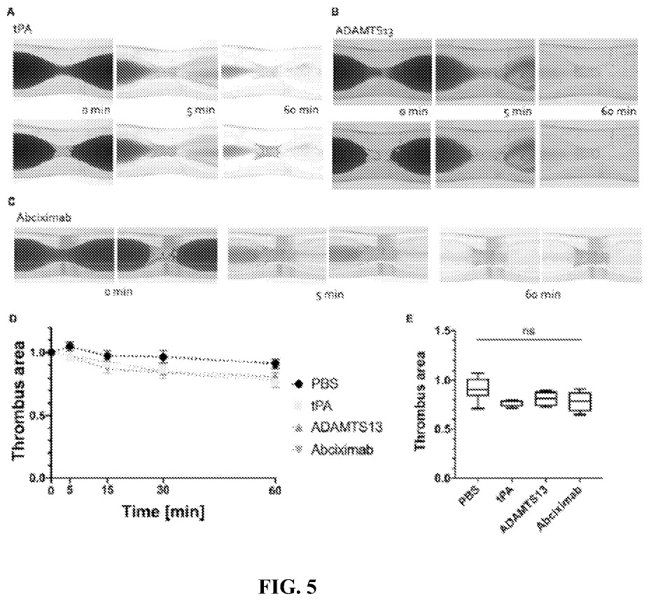

Perfusion with phosphate-buffered saline (PBS; negative control) for 60 min reduced the thrombus surface area by 9±12% (FIG. 2C). Perfusion for 60 minutes with 0.02 mg/ml tPA reduced the thrombus surface area by 23±4% (FIG. 5A), demonstrating a weak thrombolytic effect relative to DiNAC. Perfusion with ADAMTS-13 reduced the thrombus area by 19±8% (FIG. 5B), and perfusion with Abciximab resulted in a reduction in thrombus area of 22±11% (FIG. 5C). None of these three agents were significantly different than the control (FIG. 5D-5E: tPA, p=0.43; ADAMTS-13, p=0.52; Abciximab, p=0.31).

### DiNAC Does Not Lyse Fibrin Clots.

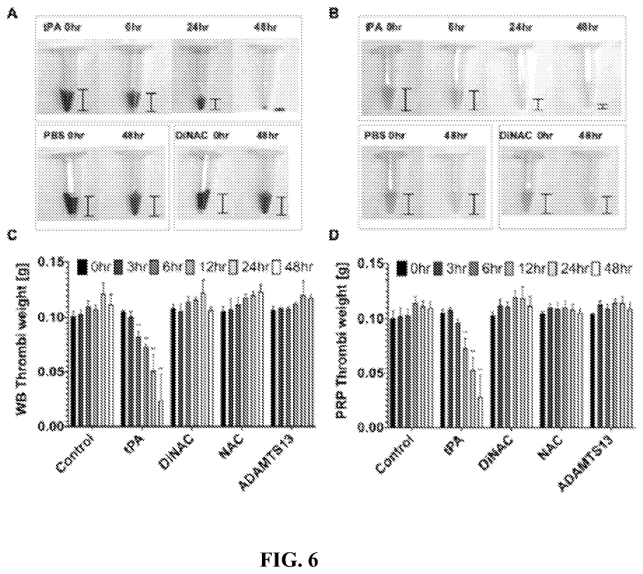

Clinical use of tPA is associated with bleeding risks due to the induction of a systemic hyper-fibrinolytic state (Crescente, M. et al. ADAMTS13 exerts a thrombolytic effect in microcirculation. Thromb Haemost 108, 527-532, 2012; Wechsler, L. R. Intravenous Thrombolytic Therapy for Acute Ischemic Stroke. New England Journal of Medicine 364, 2138-2146, 2011; Marder, V. J. Historical perspective and future direction of thrombolysis research: the re-discovery of plasmin. J Thromb Haemost 9 Suppl 1, 364-373, 2011). Fibrinous red clots were generated by re-calcifying citrated whole blood or PRP under stagnant conditions, and applied agents to quantify thrombolytic efficacy. PRP was included to determine if any platelet-specific interactions by agents may be occurring as the increase in platelet concentration creates clots with greater platelet density. Only tPA demonstrated significant thrombolytic efficacy on clots formed in these conditions (FIG. 6). tPA significantly reduced both clot volume and weight over the course of 48 h (p <0.01) compared to the control. In contrast, the other agents (DiNAC, NAC, and ADAMTS-13) did not cause any reduction in clot size nor volume. Abciximab was expected to have a less bleeding risk unless used with anticoagulation therapy (The Abciximab in ischemic Stroke Investigators. Abciximab in Acute Ischemic Stroke. Stroke 31, 601-609, 2000) and excluded due to a limited supply. Coagulated red clots generally form under low shear rate conditions such as in veins or bleeding. The lack of lytic efficacy on clots formed under stagnant conditions suggests that DiNAC may mitigate the life-threatening risk of hemorrhage associated with current tPA thrombolytic therapy.

### Platelet-Rich Thrombi Elongate and Break During DiNAC Thrombolysis.

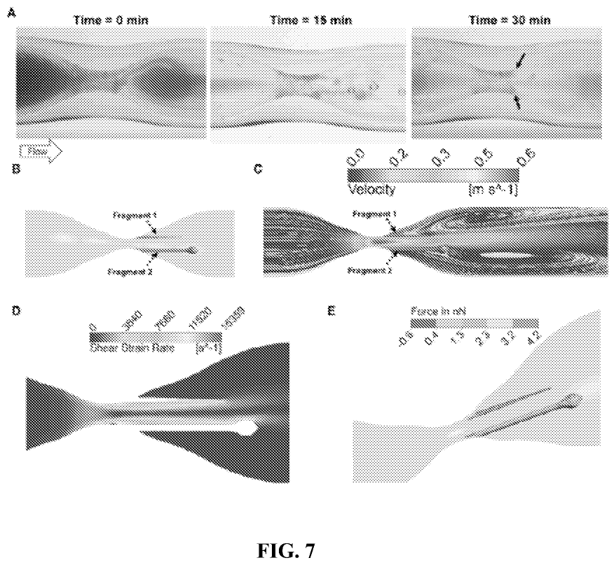

As illustrated in FIG. 7, treatment with DiNAC lyses the intact white thrombus by causing it to break apart in fragments. The fragments were typically towards the center of the lumen, and away from the wall, and often remained tethered to the main body of the thrombus. The string-like tails stretched, yet in some cases persisted for several minutes, before eventual breakage, releasing the fragments to wash downstream (FIG. 7A). The flow conditions during the thrombi lysis via fragmentation were modeled using computational fluid dynamics (CFD) (FIG. 7B) to quantify the shear stresses and drag forces on the fragments. High-velocity jet-like flow was seen in the stenosis with recirculation downstream (FIG. 7C). The elongated thrombi and fragment tails are visualized in gray in FIG. 7C. Shear rate was maximal at the stenosis reaching over 15,000 s⁻¹ while shear rates of approximately 4,000-8,000 s⁻¹ acted on the surface of thrombus downstream (FIG. 7D). The total drag force was 380 nN for a simulated thinner fragment and 780 nN for a simulated larger fragment (FIG. 7E). Thus, the net attachment force of the tethers needs to be greater than 380,000 pN, suggesting that the thrombus is held by many thousands of bonds.

### Discussion

As illustrated in this example, DiNAC, but not NAC, demonstrated the ability to completely lyse platelet-rich thrombi under perfusion in an arterial setting. These thrombi formed in the setting of arterial shear rates over fibrillar collagen are composed of VWF and platelets (Cadroy, Y., Horbett, T. A. & Hanson, S. R. Discrimination between platelet-mediated and coagulation-mediated mechanisms in a model of complex thrombus formation in vivo. J Lab Clin Med 113, 43 6-448, 1989; Para, A., Bark, D., Lin, A. & Ku, D. Rapid platelet accumulation leading to thrombotic occlusion. Ann Biomed Eng 39, 1961-1971, 2011; Crescente, M. et al. ADAMTS13 exerts a thrombolytic effect in microcirculation. Thromb Haemost 108, 527-532, 2012; Wechsler, L. R. Intravenous Thrombolytic Therapy for Acute Ischemic Stroke. New England Journal of Medicine 364, 2138-2146, 2011; Marder, V. J. Historical perspective and future direction of thrombolysis research: the re-discovery of plasmin J Thromb Haemost 9 Suppl 1, 364-373, 2011; Casa, L., Gillespie, S., Meeks, S. & Ku, D. Relative contributions of von Willebrand factor and platelets in high shear thrombosis. Journal of Hematology & Thromboembolic Diseases 4, 2016); Muia, J. et al. Allosteric activation of ADAMTS13 by von Willebrand factor. Proc Natl Acad Sci USA 111, 18584-18589, 2014); Denorme, F. et al. ADAMTS13-mediated thrombolysis of t-PA-resistant occlusions in ischemic stroke in mice. Blood 127, 2337-2345, 2016; Coulter Stephanie, A. et al. High Levels of Platelet Inhibition With Abciximab Despite Heightened Platelet Activation and Aggregation During Thrombolysis for Acute Myocardial Infarction. Circulation 101, 2690-2695, 2000; Kwon, O. K. et al. Intraarterially Administered Abciximab as an Adjuvant Thrombolytic Therapy: Report of Three Cases. American Journal of Neuroradiology 23, 447, 2002; Chen, J. eta!. N-acetylcysteine reduces the size and activity of von Willebrand factor in human plasma and mice. J Clin Invest 121, 593-603, 2011; Martinez de Lizarrondo, S. et al. Potent Thrombolytic Effect of N-Acetylcysteine on Arterial Thrombi. Circulation 136, 646-660, 2017; Hastings, S. M. & Ku, D. N. Dissolution of Platelet-rich Thrombus by Perfusion of N-acetyl Cysteine. Research and Practice in Thrombosis and Haemostasis 1, 1-145 1, 2017; Wagberg, M. et al. N,N′-diacetyl-L-cystine (DiNAC), the disulphide dimer of N-acetylcysteine, inhibits atherosclerosis in WHHL rabbits: evidence for immunomodulatory agents as a new approach to prevent atherosclerosis. J Pharmacol Exp Ther 299, 76-82, 2001; Pettersson, K. & Bergstrand, H. The antiatherogenic effect of DiNAC: experimental findings supporting immunomodulation as a new treatment for atherosclerosis related diseases. Cardiovasc Drug Rev 21, 119-132, 2003; de Lizarrondo, S. M. et al. Potent Thrombolytic Effect of N-Acetylcysteine on Arterial Thrombi. Circulation 136, 646-660, 2017; Tersteeg, C. et al. N-acetylcysteine in preclinical mouse and baboon models of thrombotic thrombocytopenic purpura. Blood 129, 1030-1038, 2017; Ku, D. N. & Flannery, C. J.

Development of a flow-through system to create occluding thrombus. Biorheology 44, 273-284, 2007). Other agents, such as tPA, ADAMTS-13, and abciximab showed a limited ability to achieve VWF-platelet thrombus lysis, and did not reduce thrombus surface area any more than the phosphate-buffered saline (PBS) control. tPA achieves thrombolysis by converting plasminogen to plasmin, which in turn, cleaves fibrin. This process requires the presence of endogenous plasminogen and takes some time in the local setting for the kinetics of biochemical reactions to occur (Hoylaerts, M., Rijken, D. C., Lijnen, H. R. & Collen, D. Kinetics of the activation of plasminogen by human tissue plasminogen activator. Role of fibrin. Journal of Biological Chemistry 257, 2912-2919 (1982); Piebalgs, A. eta!. Computational Simulations of Thrombolytic Therapy in Acute Ischemic Stroke. Scientific Reports 8, 15810, 2018). Therefore, there is concern regarding the efficacy of tPA against VWF-platelet white clots where fibrin may be less structural to the clot, and in the arterial setting where high flow rates may inhibit enzymatic reactions due to the dominance of convection. Indeed, tPA has produced mixed results against ischemic strokes from clinical arterial occlusions (Kim, E. Y. et al. Prediction of thrombolytic efficacy in acute ischemic stroke using thin section noncontrast CT. Neurology 67, 1846, 2006). Post-analysis of occlusive thrombi from patients have been unable to determine a correlation between origin and composition. Marder et al. examined 25 thrombi retrieved from the cerebral circulation of patients and did not find consistency among the suspected etiologies and compositions, but they demonstrated a large variety of structural components (Bivard, A., Lin, L. & Parsonsb, M. W. Review of stroke thrombolytics. J Stroke 15, 90-98, 2013; Marder, V. J. et al. Analysis of thrombi retrieved from cerebral arteries of patients with acute ischemic stroke. Stroke 37, 2086-2093, 2006). However, because of flow cessation, coagulation cascades that may not have caused the occlusive event are likely triggered post-occlusion in the vicinity of the original culprit thrombus.

Some lysis of VWF- and platelet-rich thrombi was observed under tPA perfusion. tPA may lyse these thrombi more efficaciously with the addition of circulating plasminogen to the system. Tersteeg et al. suspected that plasmin is a possible back-up enzyme for ADAMTS-13. They found that plasmin indeed possesses some ability to degrade platelet-VWF complexes (Tersteeg, C. et al. Plasmin cleavage of von Willebrand factor as an emergency bypass for ADAMTS13 deficiency in thrombotic microangiopathy. Circulation 129, 1320-1331, 2014). Thus, it was surprising that ADAMTS-13 did not seem to have a thrombolytic effect on the occlusive thrombi. ADAMTS-13 is a metalloprotease that reduces VWF adhesion by cleavage of ultra-large VWF multimers (Crescente, M. et al. ADAMTS13 exerts a thrombolytic effect in microcirculation. Thromb Haemost 108, 527-532, 2012; Gurevitz, O. et al. Recombinant von Willebrand factor fragment AR545C inhibits platelet aggregation and enhances thrombolysis with rtPA in a rabbit thrombosis model. Arterioscler Thromb Vasc Biol 18, 200-207, 1998) and porcine VWF has same cleavage site (A2) as human VWF45. ADAMTS-13 has been shown to have some antithrombotic activity in vivo with potential as a thrombolytic agent (Crescente, M. et al. ADAMTS13 exerts a thrombolytic effect in microcirculation. Thromb Haemost 108, 527-532, 2012; Denorme, F. et al. ADAMTS13-mediated thrombolysis of t-PA-resistant occlusions in ischemic stroke in mice. Blood 127, 2337-2345, 2016). ADAMTS-13 may require the VWF molecule be under tension to work. In the static clot, the normal length VWF may not be forcefully stretched for ADAMTS-13 cleavage (Aponte-Santamarla, C. et al. Force-Sensitive Autoinhibition of the von Willebrand Factor Is Mediated by Interdomain Interactions. Biophysical Journal 108, 2312-2321, 2015; Gogia, S. & Neelamegham, S. Role of fluid shear stress in regulating VWF structure, function and related blood disorders. Biorheology 52, 319-335, 2015). Steric hindrance might also prevent penetration of ADAMTS-13 (molecular weight 190 kDa) into the thrombus compared with a small molecule like DiNAC (molecular weight 324 Da). In a study by Crescente et al., both tPA and ADAMTS-13 were shown to reduce thrombus size (by 53.2% and 62.3%, respectively) after a 60 min treatment (Crescente, M. et al. ADAMTS13 exerts a thrombolytic effect in microcirculation. Thromb Haemost 108, 527-532, 2012). That model used the FeCl3 injury to induce the thrombus in vivo and also involved treatment with a higher concentration of ADAMTS-13 (4 g/mL), which may account for the differences in our results. Our trials were limited in concentration by the level of ADAMTS-13 in normal plasma (1 g/mL) (Soejima, K. et al. Analysis on the Molecular Species and Concentration of Circulating ADAMTS13 in Blood. The Journal of Biochemistry 139, 147-154, 2006) due to the prohibitively high cost of ADAMTS-13. The cost could ultimately limit its clinical use. Abciximab has been shown to have a positive effect as an adjuvant to thrombolytic therapy, as it inhibits the heightened platelet activation and aggregation observed in patients treated with tPA (Coulter Stephanie, A. et al. High Levels of Platelet Inhibition With Abciximab Despite Heightened Platelet Activation and Aggregation During Thrombolysis for Acute Myocardial Infarction. Circulation 101, 2690-2695, 2000; Kwon, O. K. et al. Intraarterially Administered Abciximab as an Adjuvant Thrombolytic Therapy: Report of Three Cases. American Journal of Neuroradiology 23, 447, 2002). Here, a significant thrombolytic effect with abciximab alone was not observed. Combining abciximab together with other agents may be useful.

NAC is known to reduce mucin multimers, and VWF is strikingly similar to mucins in structure (Chen, J. et al. N-acetylcysteine reduces the size and activity of von Willebrand factor in human plasma and mice. J Clin Invest 121, 593-603, 2011; Perez-Vilar, J. & Hill, R. L. The structure and assembly of secreted mucins. J Biol Chem 274, 31751-31754, 1999). NAC is currently used as a treatment for chronic obstructive lung disease and acetaminophen overdose. Chen et al. demonstrated VWF degradation by NAC (Chen, J. et al. N-acetylcysteine reduces the size and activity of von Willebrand factor in human plasma and mice. J Clin Invest 121, 593-603, 2011). De Lizarrondo et al. showed the potential use of NAC as a thrombolytic agent using an in vivo mice model (Martinez de Lizarrondo, S. et al. Potent Thrombolytic Effect of N-Acetylcysteine on Arterial Thrombi. Circulation 136, 646-660, 2017), but they found limited reperfusion with NAC treatment alone (<40%).

The experiments presented here showed high thrombolytic efficacy of DiNAC. The marked ability of DiNAC, and not NAC, to lyse platelet thrombi is a surprising discovery. DiNAC is a disulfide dimer of two NAC monomers, that has previously been studied for its anti-atherosclerotic effects (Wagberg, M. et al. N,N′-diacetyl-L-cystine (DiNAC), the disulfide dimer of N-acetylcysteine, inhibits atherosclerosis in WHHL rabbits: evidence for immunomodulatory agents as a new approach to prevent atherosclerosis. J Pharmacol Exp Ther 299, 76-82 (2001); Pettersson, K. & Bergstrand, H. The antiatherogenic effect of DiNAC: experimental findings supporting immunomodulation as a new treatment for atherosclerosis related diseases. Cardiovasc Drug Rev 21, 119-132, 2003). As detailed above, DiNAC showed a dose-response effect for concentrations of 0.02, 0.2, and 2 mM, but showed slightly decreased thrombolytic efficacy at 20 mM. The 20 mM DiNAC solution was very acidic and had high variability in efficacy. The variability was attenuated by neutralization. Thrombolysis by DiNAC created macroscopic fissures in the thrombus body, followed by the formation of tethered fragments and finally an eventual break with tolerable micro emboli passing downstream. The initial fracture is away from the wall, suggesting that the point of lysis is not a collagen bond. The strings are presumably VWF since the length is on the order of a millimeter. We quantified the drag force on thrombi fragments to be between 380 and 780 nN. To hold the thrombi in place would require approximately 3,800 to 7,800 bonds from either Gplba (Yago, T. et al. Platelet glycoprotein Ibalpha forms catch bonds with human WT vWF but not with type 2B von Willebrand disease vWF. J Clin Invest 118, 3195-3207, 2008) or GpIIb/IIIa at ˜100 pN/bond, consistent with our prior estimates of the number of bonds need to grow the thrombus (Wellings, P. J. & Ku, D. N. Mechanisms of platelet capture under very high shear.

### Cardiovascular Engineering and Technology 3, 161-170, 2012).

Clots may form via different mechanisms, thus with different resulting morphologies, and different relative content of fibrin versus VWF. Thus, different types of clot may require different thrombolytic agents. Hyper-fibrinolytic states induced by thrombolytic treatment are associated with increased mortality in many disease etiologies. Applicant therefore performed studies of in vitro red clot lysis under static conditions over 48 h with the same agents. Only tPA showed significant thrombolytic efficacy on fibrin clots, dissolving the clot with an approximate front speed of 3.5 μm/min that is comparable to the other in vitro fibrinolytic assays (Bannish, B. E., Chernysh, I. N., Keener, J. P., Fogelson, A. L. & Weisel, J. W. Molecular and Physical Mechanisms of Fibrinolysis and Thrombolysis from Mathematical Modeling and Experiments. Scientific Reports 7, 6914, 2017; Tasci, T. O. et al. Enhanced Fibrinolysis with Magnetically Powered Colloidal Microwheels. Small 13, 2017). The other agents, including DiNAC, did not show any significant red clot lysis. Thus, DiNAC may not cause severe bleeding, which has limited the use of tPA in patients due to iatrogenic hyperfibrinolysis. The limited effect of DiNAC on PRP clots, which would have greater platelet concentration than those formed with WB, suggests that DiNAC is not reacting with platelets, but may be interacting with VWF or the VWF-platelet bond to achieve thrombolysis.

As detailed above, heparin was used to block coagulation in whole blood samples used for creation of white clots to minimize the cross-effects against VWF and platelets seen frequently with citrate. However, heparin is an indirect thrombin inhibitor, and therefore allows high shear platelet-rich thrombi to form uninhibited but may have other small downstream effects on clot formation. Porcine whole blood was used in this experiment and may have species differences with human whole blood, though we expect these to be minimal (Mehrabadi, M., Casa, L. D., Aidun, C. K. & Ku, D. N. A predictive model of high shear thrombus growth. Annals of Biomedical Engineering 44, 2339-2350 (2016).

In conclusion, DiNAC, and not NAC, was highly efficacious in the lysis of VWF-platelet-rich thrombi created in a stenotic coronary artery analog system perfused under high shear stress. Other agents demonstrated a limited ability to lyse arterial thrombi in this setting, including tPA and ADAMTS-13. DiNAC was unable to lyse red fibrinous clot in a stagnant setting, while tPA was highly efficacious in the latter system. These results indicate the possibility of DiNAC as an effective thrombolytic agent against arterial occlusions, with the potential to mitigate life-threatening side effects of hemorrhage associated with current thrombolytic therapies.

### Example 3

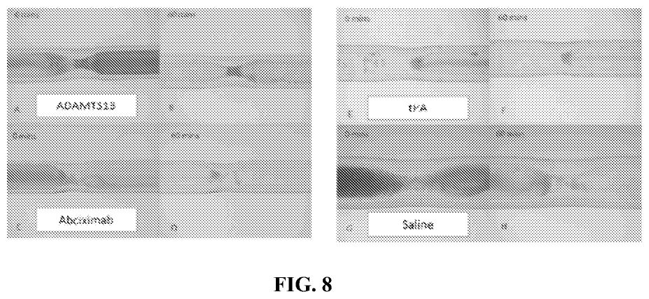

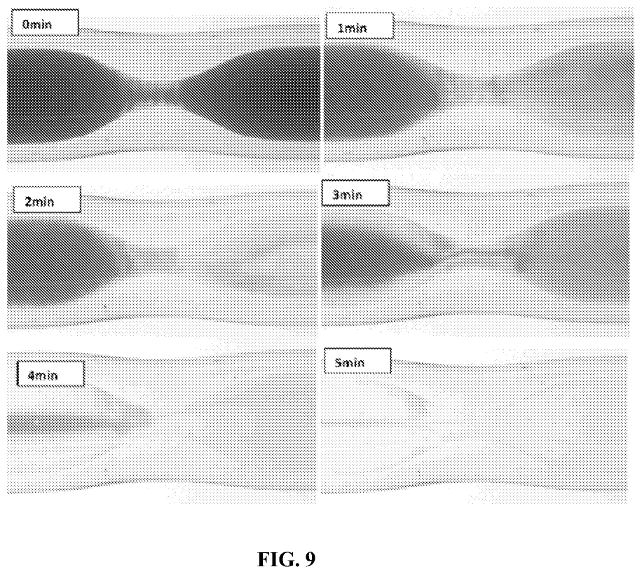

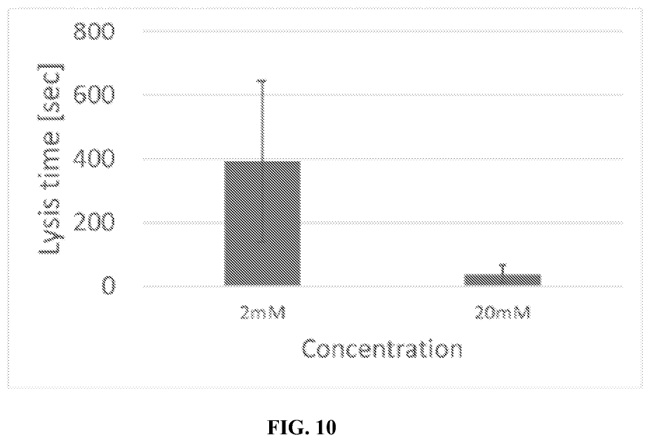

In this example, the thrombolytic activity of DiNAC and other lytic agents was tested, using the procedures described in Examples 1 and 2. As shown in FIG. 9, DiNAC was highly effective at causing lysis of white clots (sequence of clot breakup over 10 minutes). In comparison, ADAMTS-13, abciximab, NAD and saline show minimal effects with no perfusion at 1 hour (FIG. 8). Further, as shown in FIG. 10, DiNAC showed a dose response effect as a lytic agent on white thrombus.

### Example 4

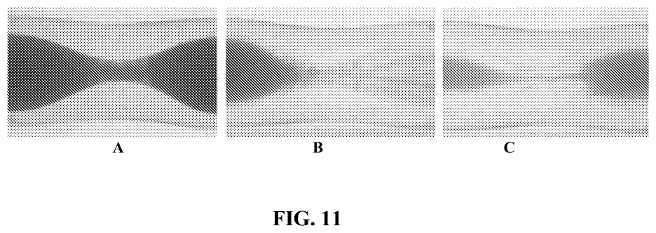

In this example, an in-vitro randomized trial was performed using heparinized human blood in collagen-coated glass capillary tubes with a designed stenosis, mimicking arterial disease. Heparinized human blood was then infused through 3 mm capillary tubes at a constant flow rate, during which near-occlusive thrombi were formed at the region of stenosis (FIG. 11). DiNAC was then infused at a constant flow rate through the capillary tubes in order to observe thrombolytic activity, using a combination of live pressure monitoring and microscopic image capture. All infusions were control-matched with a phosphate-buffered saline solution in lieu of DiNAC.

Human whole blood formed platelet-rich thrombi with the high shear region of a collagen coated stenosis. This thrombus occluded the 3 mm lumen of a high-grade stenosis, effectively stopping blood flow in approximately 5 minutes. Ensuing perfusion of 20 mM DiNAC caused complete lysis of the white clot within 10 minutes while perfusion of tPA or ADAMTS-13 did not alter the clots or restore flow (p<0.01).

Thus, DiNAC is able to cause thrombolysis of acute clots formed via the infusion of human blood through collagen-coated stenotic capillary tubes.

Various modifications of the invention, in addition to those described herein, will be apparent to those skilled in the art from the foregoing description. Such modifications are also intended to fall within the scope of the appended claims. Each reference cited in the present application, including all patents, patent applications, and non-patent literature, is incorporated by reference in its entirety.

1. A method comprising presenting an area with cystine, or a pharmaceutically acceptable salt or derivative thereof;
wherein the area is selected from the group consisting of an area within a subject, a cavity, a device, and a combination thereof; and
wherein the method is selected from the group consisting of:
a method of treating or preventing thrombus formation in the subject in need thereof by administering to the subject the cystine, or the pharmaceutically acceptable salt or derivative thereof, in an amount effective to induce thrombolysis in the subject;
a method of treating or preventing a disease or disorder associated with thrombus formation in the subject in need thereof by administering to the subject a therapeutically effective amount of the cystine, or the pharmaceutically acceptable salt or derivative thereof; and
a method of treating or preventing thrombus formation in the cavity or the device by contacting the cavity or the device with the cystine, or the salt or derivative thereof.

2. (canceled)

3. The method of claim 1, wherein the thrombus comprises at least trace amounts of von Willebrand factor.

4. The method of claim 1, wherein the thrombus substantially comprises von Willebrand factor and platelet cells, wherein the platelet cells are present at a concentration of greater than about 5%.

5. The method of claim 1, wherein the thrombus is substantially free of red blood cells.

6. The method of claim 1, wherein the thrombus is substantially free of fibrin.

7. The method of claim 1, wherein the cystine is selected from the group consisting of N,N′-diacetyl-L-cystine and N,N′-diacetyl-D-cystine.

8. (canceled)

9. The method of claim 1, wherein the cystine is substantially pure.

10. The method of claim 1, wherein the cystine is substantially free of N-acetylcysteine.

11. The method of claim 1, wherein the cystine is administered as a liquid dosage form.

12. The method of claim 1, wherein the cystine is administered to the subject intravenously.

13. The method of claim 1, wherein the cystine is administered as an oral dosage form.

14. The method of claim 13, wherein the oral dosage form is a tablet or a liquid.

15. The method of claim 1, wherein the cystine is provided as a solution having a pH in the range of about 5 to about 8.

16. (canceled)

17. The method of claim 1, wherein the cystine is administered at a concentration of about 0.5 mM to about 50 mM.

18.-20. (canceled)

21. The method of claim 1, wherein the cystine is administered in combination with a lytic agent.

22. The method of claim 21, wherein the lytic agent is selected from the group consisting of: tissue plasminogen activator (tPA), ADAMTS-13, abciximab and N-acetyl cysteine (NAC).

23. The method of claim 1, wherein the subject is a human subject.

24. The method of claim 1, wherein the subject is suffering from a disease or disorder selected from the group consisting of: stroke, myocardial infraction, leg ischemia, a sickle-cell anemia, Disseminated Intravascular Coagulation, extracorporeal circulation, heart failure, valvular disease, aortic stenosis, and venous thrombosis.

25. The method of claim 1, wherein the thrombus formation occurs in an artery of the subject selected from the group consisting of a carotid artery and a coronary artery.

26. (canceled)

27. The method of claim 1, wherein the treating or preventing results in a reduction of diameter of the thrombus of at least about 50%.

28.-30. (canceled)

31. The method of claim 1, wherein the disease or disorder is selected from the group consisting of stroke, myocardial infraction, leg ischemia, a sickle-cell anemia, Disseminated Intravascular Coagulation, extracorporeal circulation, heart failure, valvular disease, aortic stenosis, and venous thrombosis.

32.-45. (canceled)

46. The method of claim 1, wherein the cystine is provided as a solution having a pH of about 7.

47. (canceled)

48. The method of claim 1, wherein the cystine is administered at a concentration of about 2 mM to about 20 mM.

49. The method of claim 1, wherein the cystine is administered at a concentration of about 3 mM to about 10 mM.

50. The method of claim 1, wherein the cystine is administered at a concentration of about 10 mM.

51.-56. (canceled)

57. The method of claim 1, wherein the treating or preventing results in a reduction of diameter of the thrombus of at least about 70%.

58. The method of claim 1, wherein the treating or preventing results in a reduction of diameter of the thrombus of at least about 95%.

59.-60. (canceled)

61. The method of claim 1, wherein the cavity or device comprises tubing, a valve, a graft, a circuit, a stent, a catheter, or a thrombectomy device

62. The method of claim 61, wherein the tubing is a blood tubing.

63. The method of claim 61, wherein the valve is a heart valve.

64. The method of claim 61, wherein the graft is a dialysis graft.

65.-85. (canceled)
